(19) United States
(12) Patent Application Publication
(10) Pub. No.: US 2004/0091579 A1
(43) Pub. Date: May 13, 2004

(54) Extruded foodstuffs having maintenance level actives

(75) Inventors: Gordon R. Huber, Sabetha, KS (US); David R. Jones, Palm Beach, FL (US); John C. Kuenzi, Bern, KS (US); Kevin D. Kuenzi, Perry, KS (US); Francisco A. Cabrera, Overland Park, KS (US)

Correspondence Address: HOVEY WILLIAMS LLP, 2405 GRAND BLVD., SUITE 400, KANSAS CITY, MO, 64108, US

(73) Assignee: RUBICON SCIENTIFIC LLC;

(21) Appl. No.: 10/294,057

(22) Filed: Nov. 13, 2002

(51) Int. Cl. A61K 47/00; A23L 1/31; A23L 1/315

(52) U.S. Cl. 426/72; 426/105; 424/439

(57) ABSTRACT

Improved extruded human and animal foodstuff products are provided which include nutritional or therapeutically effective amounts of an active such as vitamins, vitamin precursors, and drugs such as tetracycline. The products are produced by extrusion, preferably with initial preconditioning followed by extrusion through an extruder equipped with a die assembly having an elongated, tubular body and an endmost restricted orifice die plate.

### BACKGROUND OF THE INVENTION

1. Field of the Invention

The present invention is broadly concerned with improved, extrusion-processed foodstuffs having enhanced nutritional and/or therapeutic properties. More particularly, the invention pertains to such foodstuffs (e.g., rice) and methods of producing the same, wherein the products include relatively minor but effective amounts of an added, non-naturally occurring active such as vitamins, a vitamin precursor or drug(s). If desired, the actives may be initially encapsulated with fat or other encapsulant prior to extrusion processing with foodstuffs.

2. Description of the Prior Art

Supplementation of naturally occurring or processed human foods or animal feeds with vitamins, minerals or other nutraceuticals has long been practiced. For example, it is common to add vitamins to a variety of foods such as dairy products. Additionally, it has been suggested in the past that food products may be a suitable vehicle for administration of drugs and medicaments. A problem with this approach, however, is that the added actives are often used at relatively high levels, in order to insure that the active is present over an extended period. As a consequence, when such a food product is ingested, the active is initially present at a relatively high level in the blood stream, and then gradually tapers in concentration.

For example, rice is eaten on a daily basis by about 80% of the human population, and in many instances rice is the staple of human diets. Also, rice is sometimes used as a part of animal feeds, particularly high-quality pet feeds. While rice has many excellent nutritional qualities, it is known to be deficient in vitamin A. Accordingly, a number of efforts have been made towards enhancing the vitamin A content of rice, through traditional plant breeding and more sophisticated genetic manipulations. However, the goal of enhanced vitamin A rice while maintaining necessary other nutritional benefits and taste and eating qualities has not been entirely met.

Many humans and animals require or benefit from drug treatments. In traditional practice drugs are administered in a variety of ways, such as by injection or through ingestion. The latter course is often preferred, and for human treatment is usually acceptable. However, it is sometimes very difficult to induce an animal to swallow a drug, particularly where the drug (e.g., tetracycline) has a bitter or otherwise offensive taste. It has been suggested in the past to supplement food products with vitamins, minerals and other active ingredients such as nutraceuticals. However, the formulation of enhanced rice products including vitamin A (or precursors thereof such as β-carotene) or drugs has not been successfully accomplished.

### SUMMARY OF THE INVENTION

The present invention overcomes the problems outlined above and provides methods (and final products) wherein a starting composition including a foodstuff is extrusion-processed with the addition of an active selected from the group consisting of vitamins, minerals, vitamin precursors and/or drugs. In this fashion, final foodstuff products having enhanced nutritional or therapeutic properties are obtained. In one aspect of the invention, rice products are formulated with a vitamin A precursor, namely β-carotene, at a level from about 0.005-0.5% by weight, more preferably from about 0.008-0.2% by weight, based upon the total weight of the product, dry basis. More broadly, actives in accordance with the invention are typically added to extrusion-processed foodstuffs at a level of from about 0.0000001-2% by weight, more preferably from about 0.000001-0.5% by weight of the product, dry basis.

In the extrusion process, a starting composition comprising one or more foodstuffs is passed into and through an elongated extruder barrel having an internal, elongated, axially rotatable, helically flighted screw with an endmost extrusion die. In this fashion, the starting composition is subjected to elevated temperature, pressure and shear for cooking thereof. The desired active may be added to the initial starting composition or during processing thereof. Preferably, the starting composition is preconditioned before extrusion, which involves moisturizing the starting composition through addition of steam and/or water with mixing to effect partial precooking.

Although a variety of different types of extrusion systems can be used in the processes of the invention, those described in PCT Publication WO 00/08945 (incorporated by reference herein) are particularly preferred. Broadly speaking, such apparatus includes an elongated extruder having a tubular barrel with an outlet end and at least one (and preferably a pair of) elongated, axially rotatable helically flighted screw(s) within the barrel for moving material therethrough. A tubular die assembly is operatively coupled to the barrel adjacent the outlet end thereof and has an elongated, tubular body with an apertured die secured to the end of the tubular body remote from the extruder barrel; the die opening(s) are substantially smaller in cross-sectional area than the minimum cross-sectional area of the barrel and tubular body.

Preferably, the apparatus is designed so that the residence or retention time of the product within is greater than the residence time within the extruder, and specifically a retention time ratio of the die assembly retention time to extruder retention time of at least about 5, and preferably from about 7-15, is established. The absolute retention time of the product within the extruder usually ranges from about 3-20 seconds whereas the retention time within the die assembly is at least about 15 seconds, and more preferably from about 50-600 seconds.

Another factor in the preferred apparatus is selection of an appropriate die assembly internal volume/extruder free volume ratio. The free volume of the extruder is calculated as the total internal volume of the extruder barrel minus the volume occupied by the screw(s) therein. The internal volume of the die assembly less any components therein is also determined. Broadly, the die assembly internal volume/extruder free volume ratio should be at least about 2, preferably from about 3-20, and more preferably from about 3-6.

When processing foodstuff products in accordance with the invention, maximum pressure levels achieved in the extruder barrel should be on the order of from about 50-2000 psi, and more preferably from about 100-600 psi. Maximum material temperature within the barrel is up to about 150° C. and more preferably from about 60-110° C. The internal screws within the barrel should be operated at rotation speeds of at least about 300 rpm and more preferably from about 400-1500 rpm. When a preconditioner is used, the foodstuff composition leaving the preconditioner should have a total moisture content of from about 6-50% by weight, and more preferably from about 18-35% by weight, based upon the total weight of the preconditioned mixture taken as 100% by weight. The residence time in the preconditioner is preferably at least about 15 seconds, more preferably from about 15-600 seconds, and more preferably from about 120-240 seconds. The maximum temperature in the preconditioner should range from about 30-100° C., and more preferably from about 85-95° C.

Upon emergence from the extruder, the foodstuff material enters the die assembly and passes through the tubular die body and final restricted orifice die plate. The temperature and pressure conditions within the die assembly should be: temperature, up to about 150° C., and more preferably from about 85-105° C.; and pressure, from about 50-2000 psi, and more preferably from about 100-600 psi. Upon emerging from the die plate, the extrudate should have a moisture content of from about 10-70% by weight, and preferably from about 20-50% by weight, based upon the total weight of the extrudate taken as 100% by weight. Generally, the extruded product is dried by any convenient means to a final moisture content of from about 2-14% by weight.

### DETAILED DESCRIPTION OF THE PREFERRED EMBODIMENT

The following examples set forth presently preferred techniques for the production of foodstuff products. It is to be understood, however, that these examples are provided by way of illustration and nothing therein should be taken as a limitation upon the overall scope of the invention.

### EXAMPLE 1

In this example, an extruded rice product was prepared with β-carotene addition, in order to increase the vitamin A nutritional potential of the rice. Two extrusion runs were carried out, a control and a run with β-carotene addition at a level to achieve 6000 μg β-carotene per 50 g of dry rice.

The extrusion system included a Wenger TX57 twin screw extruder with a Wenger Model 2 DDC preconditioner. The extruder barrel was equipped with an elongated tubular die adaptor and an endmost, restricted orifice die as described and illustrated in PCT Publication No. WO 00/08945, incorporated by reference herein. The extruder barrel and the spacer were externally jacketed, with hot oil circulating through the external jackets for indirect temperature control. The product emerging from the die was cut using a conventional motor-driven cutting knife assembly with Teflon-coated knife blades. Thereupon, the product was dried and collected in tubs, held 1-2 hours, and then passed through a cooler.

Control Run #1 employed a starting mixture made up of 99.25% by weight long rice flour and 0.75% by weight Dimodan PVK surfactant, In Run #2, the starting ingredient mixture contained 98.77% by weight long rice flour, 0.75% Dimodan PVK and 0.48% by weight Betaten 2.5% WDP β-carotene (Cognis).

The following Table 1 sets for the conditions in these illustrative runs.

TABLE 1

| | | Run #1 | Run #2 |
|---|---|---|---|
| DRY RECIPE INFORMATION | | | |
| Feed Screw Speed | rpm | 19 | 19 |
| PRECONDITIONING INFORMATION | | | |
| Preconditioner Speed | rpm | 150 | 150 |
| Steam Flow to Preconditioner | kg/hr | 40 | 40 |
| Water Flow to Preconditioner | kg/hr | 35.58 | 31.74 |
| Preconditioner Discharge Temp. | ° F. | 214 | 214 |
| Moisture Entering Extruder | % wb | 35.58 | 33.75 |
| EXTRUSION INFORMATION | | | |
| Extruder Shaft Speed | rpm | 160 | 160 |
| Extruder Motor Load | % | 21 | 22 |
| Control/Temperature 1st Head | ° C. | HO/90/90 | HO/90/90 |
| Control/Temperature 2nd Head | ° C. | HO/90/90 | HO/90/90 |
| Die Spacer Temperature | ° C. | HO/90/87 | HO/90/88 |
| Head/Pressure (2^{nd} Head) | kPa | 1034.2 | 1379 |
| Die Spacer Pressure | psi | 300 | 350 |
| Knife Drive Speed | rpm | 47 | 47 |
| FINAL PRODUCT INFORMATION | | | |
| Extruder Discharge Moisture | % wb | 38.5 | 33.37 |
| Extruder Discharge Rate | kh/hr | 162 | 162 |
| Dryer Discharge Moisture | % wb | 14.74 | 13.81 |
| Extruder Performance | | Stable | Stable |

The product from Run #2 was in every way an acceptable rice, cooked well, and had no perceptible taste problems. This product had the appearance of naturally occurring or native rice. In general, where such appearance is desired, the extruded product should be in the form of grains having an average length of from about 0.25-0.75 inches, an average diameter of from about 0.025-0.125 inches, and a generally uniform, white color.

### EXAMPLE 2

In this example, rice products were formulated containing a human/animal drug (Tetracycline) and an animal heartworm drug (ivermectin).

The equipment and general process employed were the same as in Example 1. The ivermectin run (#3) starting ingredients included 99.21% by weight long rice, 0.75% by weight Dimondan PVK and 0.04% by weight of an ivermectin premix. The latter was made up of 49.13% by weight propylene glycol, 45.57% by weight distilled water, 4.98% by eight Red #40 dye and 0.31% by weight of a 1% ivermectin solution (Loveland Industries, Inc.).

The run #4 starting ingredients included 96.52% by weight long grain rice, 0.75% Dimodan PVK, and 2.75% by weight oxytetracycline powder. In this case, the drug was premixed with 1.75 kg of water in a Hobart mixer.

In run #3, the tetracycline premix was added to the other ingredients before preconditioning. In run #4, the liquid ivermectin premix was pumped into the last port of the preconditioner, before entry into the extruder (flow rate 46-920 ml/hr).

The following Table 2 sets forth the recorded conditions for these runs.

TABLE 2

| | | Run #3 | Run #4 |
|---|---|---|---|
| DRY RECIPE INFORMATION | | | |
| Dry Recipe Rate | kg/hr | | 125 |
| Feed Screw Speed | rpm | 19 | 21 |
| PRECONDITIONING INFORMATION | | | |
| Preconditioner Speed | rpm | 150 | 150 |
| Steam Flow to Preconditioner | kg/hr | 50 | 50 |
| Water Flow to Preconditioner | kg/hr | 31.74 | 28.50 |
| Preconditioner Discharge Temp. | ° F. | 214 | 214 |
| Moisture Entering Extruder | % wb | 33.66 | 36.45 |
| EXTRUSION INFORMATION | | | |
| Extruder Shaft Speed | rpm | 160 | 160 |
| Extruder Motor Load | % | 22 | 17 |
| Control/Temperature 1st Head | ° C. | HO/90/90 | HO/90/90 |
| Control/Temperature 2nd Head | ° C. | HO/90/90 | HO/90/90 |
| Die Adaptor Temperature | ° C. | HO/90/88 | HO/90/88 |
| Head/Pressure (2^{nd} Head) | kPa | 1379 | 689.5 |
| Die Adaptor Pressure | psi | 350 | 250 |
| Knife Drive Speed | rpm | 47 | 47 |
| FINAL PRODUCT INFORMATION | | | |
| Extruder Discharge Moisture | % wb | 34.15 | 37.09 |
| Extruder Performance | | Stable | Stable |

It has been determined that the actives maintain their potency in the rice products of the invention, and that good reproducibility in terms of active content can be achieved via the preferred extrusion processes. In one form of the invention, the products are designed so that sufficient vitamin or drug-based active is present in the feed to establish and continuously maintain in the bloodstream of the human or animal consuming the product a desired, effective amount of the active. Thus, with β-carotene supplemented rice, it is desired to employ sufficient quantities of the active so that the final rice product has materially increased vitamin A nutritional properties. Likewise, where drug(s) are used as actives, it is often desirable to design the rice with a human or animal's daily ration needs in mind, i.e., so that if normal quantities of the rice are consumed on a daily basis, a therapeutically effective amount of the drug(s) is established and maintained in the bloodstream of the consuming human or animal. In any case, the extruded rice products hereof have at least one property (e.g., enhanced vitamin content or the presence of therapeutic drug(s)) not found in native rice.

While tetracycline and ivermectin have been specifically illustrated in Example 2, it will be understood that a wide of drugs can be used, both with rice and other foodstuffs. Such drugs include anabolic agents, analgesics, analgesic with tranquilizing effects, tranquilizers, ACE inhibitors (angiotensin converting enzyme inhibitors, anesthetics, antacids, adsorbents, anti-flatulents, anti-inflammatories, anti-convulsants, anti-diarrheals, antidotes, anti-cancer drugs, antifungals, anti-ricketsials, antihistamines, antimicrobial agents, antiprotozoal agents, antiseptics, antispasmodics, antitussives, antimalarals, antivirals, antibiotics, anthelmintics, autonomic drugs, behavior modification rugs, bloat preparations, blood products, bronchodilators and expectorants, cardiovascular drugs, coccidostats and coccidiocidals, colostrum antibodies, counterirritants, dental prpearations, diuretics, endocrine system modifiers, flea control products, growth promoters, hematinics, hemostatics, hormones and analogs, immunostimulants, laxatives, muscle relaxants, pancreatic enzymes, proviotics, respiratory stimulants, sedatives, urinary acidifiers, urinary antiseptics, uterine preparations, vitamins and minerals, allergy antigens, atibodies (immunoglobins), aquaculture biologicals, vaccines, toxoids, antitoxins, anthelmintics, ectoparasite control, endectocides (systemic parasiticides), flea control, flea, tick and pest killers, heartworm parasiticides and preventatives, larvicides, miticides, and repellents. Similarly, while vitamin A is a preferred vitamin active in the case of rice, other vitamins and/or minerals can also be used in rice or otherwise. Representative actives include biotin, calcium, chloride, chromium, cobalt, copper, fluoride, iodine, iron, magnesium, manganese, molybdenum, phosphorus, potassium, sodium, sulfur, vitamin A, vitamin B complex, vitamin B1 (thiamine), vitamin B2 (riboflavin), vitamin B3 (niacin), vitamin B5 (panthothenic acid), vitamin B6 (pyridoxine), vitamin B9 (folic acid), vitamin B12 (cobalamin), vitamin C (ascorbic acid), vitamin D, vitamin E, vitamin K, and zinc.

It is also contemplated that encapsulated actives can be used in the products of the invention. For example, certain drugs such as tetracycline have an undesirable or bitter taste. In order to mask such taste characteristics, the actives of the invention can be ground to a coarse size and encapsulated with a material such as high melting temperature fat (that is, fat which will not melt under the extrusion processing conditions but which will break down in the digestive system). Such encapsulated actives can then be incorporated into the products of the invention as outlined above.

Thus, an active may be encapsulated in any suitable encapsulant and the resultant encapsulated active may be incorporated into another product which is an extrudate. For example, an active such as tetracycline may be high temperature fat-encapsulated and then incorporated into a secondary filling or other product; this filling may then be co-extruded with a shell extrudate to create a combined product having the encapsulated active present only in the central fill. In this fashion, the unpleasant taste of tetracycline may be effectively masked, but nevertheless effective when ingested.

We claim:

1. In a method of producing an edible foodstuff including the steps of providing a starting composition comprising said foodstuff and passing the starting composition into and through an elongated extruder barrel having an elongated, axially rotatable, helically flighted screw within the barrel and an endmost extrusion die in order to subject the starting composition to elevated temperature, pressure and shear for cooking thereof, the improvement which comprises the step of adding a quantity of an active selected from the group consisting of vitamins, vitamin precursors, drugs and mixtures thereof to the starting composition prior to passage thereof through said die, said active being added at a level of from about 0.0000001-2% by weight of the composition, dry basis.

2. The method of claim 1, including the step of initially passing said starting composition through a preconditioner prior to passage thereof into and through said extruder, and adding said active to said starting composition prior to passage thereof through the preconditioner.

3. The method of claim 2, including the step of adding moisture to the starting composition during passage thereof through said preconditioner.

4. The method of claim 1, including the step of adding a surfactant to said starting composition prior to passage thereof through said extruder.

5. The method of claim 1, said foodstuff comprising rice, said active comprising β-carotene.

6. The method of claim 5, said β-carotene being added at a level of from about 0.005-0.5% by weight of the composition, dry basis.

7. The method of claim 1, said active comprising tetracycline.

8. The method of claim 1, said starting composition comprising rice flour.

9. The method of claim 1, said active being present at a level of from about 0.000001-0.5% by weight of the composition, dry basis.

10. The method of claim 1, said foodstuff selected from the group consisting of carbohydrate-bearing grain products, proteinaceous grain products, meat and meat byproducts.

11. The method of claim 10, said foodstuff selected from the group consisting of wheat, corn, soy, barley, oats, sorghum, potato, tapioca, quinoa, rice, beef, chicken, fish, pork, lamb, blood meal, bone meal, and mixtures thereof.

12. The method of claim 1, said active being encapsulated in an encapsulant.

13. A foodstuff produced by the method of claim 1.

14. An extruded rice product comprising at least partially cooked rice and having at least one added active therein selected from the group consisting of vitamins, vitamin precursors, drugs and mixtures thereof, the extruded rice product having at least one property not found in native rice.

15. The product of claim 14, said active comprising β-carotene.

16. The product of claim 15, said β-carotene being present at a level of from about 0.005-0.5% by weight of the product, dry basis.

17. The product of claim 14, said active being a drug, said drug being present at a level of from about 0.0000001-2% by weight of the product, dry basis.

18. The product of claim 14, said product having the appearance of native rice.

19. The product of claim 14, said active being encapsulated with an encapsulant.
